(19) United States
(12) Patent Application Publication
(10) Pub. No.: US 2014/0288952 A1
(43) Pub. Date: Sep. 25, 2014

(54) SYSTEM AND METHOD FOR TRACKING AND MANAGING MEDICAL DEVICE INVENTORY

(71) Applicant: Medical Tracking Solutions, Inc., Jacksonville, FL (US)

(72) Inventors: Gregory Smith, Ponte Vedra Beach, FL (US); Stacy Smith, Jacksonville, FL (US); Sebastian Rudbach, Atlantic Beach, FL (US)

(73) Assignee: MEDICAL TRACKING SOLUTIONS, INC., Jacksonville, FL (US)

(21) Appl. No.: 14/224,237

(22) Filed: Mar. 25, 2014

Related U.S. Application Data
(63) Continuation-in-part of application No. 13/050,232, filed on Mar. 17, 2011.
(60) Provisional application No. 61/389,189, filed on Oct. 1, 2010.
(60) Provisional application No. 61/340,402, filed on Mar. 17, 2010.

(51) Int. Cl. G06Q 10/08; G06Q 50/22

(52) U.S. Cl. CPC G06Q 10/087; G06Q 50/22
USPC 705/2

(57) ABSTRACT

A system and method of managing the logistics of trays and lots of medical inventory, from the manufacturer's initial release until final discharge by the end user healthcare provider. The system is driven by one or more software applications running on a central, secure server accessible by remote handheld telecommunication devices. The system comprises software modules providing functionality for the management of specific medical cases, the billing process, warehouse reconciliation of used inventory, a loaner program for borrowing otherwise unavailable inventory, and many other processes. Additional features of the system include a geographic map, an inventory list of medical devices, an imaging assembly to read barcodes or other optic identifiers, an Food and Drug Administration news assembly incorporating a live RSS news feed for releases about product defects or recalls.

### CROSS REFERENCES TO RELATED APPLICATIONS

This application is a continuation-in-part of U.S. patent application Ser. No. 13/050,232, filed Mar. 17, 2011, which claims the benefit of and priority to U.S. Provisional Patent Application Ser. No. 61/389,189, filed on Oct. 1, 2010, and to U.S. Provisional Patent Application Ser. No. 61/340,402, filed on Mar. 17, 2010, the entire contents of each of which are incorporated herein by this reference.

### BACKGROUND

(a) Field of the Invention

The invention relates generally to the field of logistics tracking of medical inventory, and more specifically, to a system and method of tracking surgical implant devices and related tools through the entire life-cycle of such devices.

(b) Description of Related Art

In the medical industry, surgical implant devices and the instruments for installing them are organized by the manufacturer into trays, and subcategorized into lots. The lots are the surgical implant devices or the medical instruments needed to install such implants. Each tray is comprised of one or more implants and/or instruments, with each tray having a unique alphanumeric identifier for inventory purposes, and each lot having a unique lot number. Each tray has an arrangement of implants or instruments predetermined by the manufacturer.

Current smartphone applications tailored to track inventory are limited to tracking shipment information of each tray. The shipping information is accessed via the courier's website, and the smart phone application acts as a reporting tool showing the user the shipping status of the shipment. These applications offer limited information, such as the time the courier received the shipment, the estimated time and address of delivery, and in some cases the location of the package during the shipping process. Notably, these applications track shipments, as opposed to trays or lots.

Other inventory logistics systems are capable of managing inventory from a handheld device, but lack integrated features to track shipping information or data relating to use of inventory. Some such inventory systems are driven by RFID tags, which creates logistics discontinuities when the radio signal is disrupted, such as by a lack of electric power.

Additionally, the prior art does not teach a comprehensive logistics or tracking system in which one can track the location of a specific medical implant during the lifecycle of the device. Thus, it can often be difficult or impossible to locate defective devices that have been recalled by regulatory authorities, such as the United States Food and Drug Administration.

Further problems with managing medical device inventory persist in the current industry. For example, under current practice the case scheduling process is susceptible to scheduling errors, lost or unavailable trays, billing problems, and documentation deficiencies. The current case scheduling process begins when a doctor contacts a medical device sales representative to schedule a surgery, indicating what trays and lots will be needed for the surgery. The representative schedules the case by adding it to his or her personal calendar, meaning that the representative's organization has no record of the case, and no oversight over its completion. The representative manually searches the inventory at the healthcare location for the needed inventory to determine what is available. The representative has no way to review the organizational inventory to determine what inventory may be available at nearby healthcare facilities. This lack of system-wide information can cause double-booking of inventory when multiple representatives unknowingly schedule inventory for medical procedures that overlap in time.

In other types of current systems, the representative has to place a telephone call to a headquarters or field inventory office to order medical inventory for surgeries booked by a hospital or doctors office. This process often required remaining on hold for lengthy periods, and after placing the order there is no assurance that another representative has not already booked the inventory needed. There is no visibility between representatives for them to see which one of them has requested which inventory. Once the inventory is shipped, the representative must monitory the tracking information for the shipment to ensure proper and timely delivery. Schedule changes for the medical procedure are often noted manually by the representatives, and in many cases the handwritten notes are inaccurate or lost. In many instances, the doctors will want to review the types of instrumentation inserted into patients on previous cases, or they have question about future cases. The representatives must manually review paper files or notes to locate the relevant information, which sometimes a trip to the nearest field office to examine x-rays and other information from previous cases.

While the medical procedure is being performed, the representative manually logs the inventory used by the doctor. At the end of the procedure, the representative prepares a manual requisition to submit to the healthcare provider for billing purposes.

In many instances, the representatives log the lots used by writing on their scrubs, their hands, note cards, and sometimes on a hospital requisition. At the conclusion of the surgery, the representative must recount the lots that were implanted into the patient. This is a very error prone process, and mistakes often arise in transferring the hand written notes to the nurse, doctor, or hospital. After the information is conveyed to the nurse, the representative delivers the paper requisition to the billing office of the facility, as well as to a purchasing office, if any. These steps can be time consuming at large health care facilities where the relevant administrative offices are located remotely from surgical rooms. These requisitions are subject to delays by the representative and the healthcare provider, meaning that payment is often delayed and even refused when accurate billing records cannot be reconstructed on hindsight after the passage of time. Finally, the representative faxes the requisition to its local field office and headquarters to replace the needed parts and begin the paperwork process with the manufacturer. During the entire process, there is no visibility of recalls, and no way to stop a surgical process in real-time for recalled implant devices.

Another problem with current medical inventory tracking systems is that these systems provide no visibility of inventory that is in the warehousing and disinfecting process. After a tray is used in a medical procedure, it is returned to the manufacturer or other facility to repair, replenish, and disinfect the trays and lots. These current systems, which are manual processes, do not track data associated for each tray. For example, the Food and Drug Administration (“FDA”) requires trays to be disinfected, which is typically done in a batch process. Under current tracking technology, there is no data repository of tray status or an audit trail for each tray. Consequently, it is virtually impossible to identify trays that are subsequently subject to FDA recalls when the FDA identifies a particular disinfecting batch as defective.

Currently, manufacturers track trays via spreadsheets and hand written documents. When a tray arrives at the warehouse, an employee charts the arrival in the spreadsheet. The tray then moves into a replenishment area where another employee reviews the integrity of the tray and its lots to determine whether there are any problems, such as nonconforming or damaged parts, or parts that simply need to be replaced. This employee then makes notes and removes the nonconforming or damaged part. After the tray is replenished, it is ready to go through washer and onto the autoclave. The tray is washed, and an employee in the warehouse documents the tray and the date and time of wash. Next, the tray is moved to the autoclave where it is sterilized. When the tray goes through the autoclave process, the machine generates an identification number or code corresponding to the autoclave batch data, such as the time the batch was performed, the temperature reached by the machine, and other relevant data. All of this data is documented on paper for corroboration in the event of an audit. Once the autoclave cycle is complete, the tray is stored in warehouse in the location where the manufacturer stores trays of that type. Most manufacturers maintain a bill of materials on paper for corroboration against the spread sheet when there set is full or missing parts. The foregoing system is substantially manual, and it is unnecessarily susceptible to error and omission due to human error.

Another problem in the industry is that present medical device management systems do not account for instances where a tray is needed, but not currently available from the manufacturer. Currently when a representative orders a tray for a surgery, a telephone call is made to the customer service representative at the headquarters, and the call typically gets forwarded to the warehouse operations staff via email or a hand written form providing notification to the warehouse that there is a representative in a certain geographic location who has requested a tray for a surgery. The operations staff then search the warehouse inventory to determine whether the requested tray is available. Additional requests from other representatives multiplies the paperwork, often leading to errors and omissions due to disorganization and confusion between requests. Once the tray is physically located in the warehouse, it is shipped to the requesting representative. This shipping event is recorded on a paper log that includes the destination of the tray, the type of tray, and any relevant tracking information.

If the requested tray is currently unavailable from the warehouse, the staff must review paper records to determine the destination of the last tray sent of that type. The staff is unable to determine whether the tray remains at the shipping destination of record, or whether the tray has been subsequently moved to a different location, such as another hospital. Sometimes the trays are eventually located, and sometimes they are not. If the tray, or a similar one, is eventually located, the warehouse staff can execute a transfer, sending the tray to the requesting representative. The warehouse staff documents this transfer via paper notes and a spreadsheet. Again, this is a time consuming and error prone process.

The present system seeks to overcome the limitations of previous systems by providing a comprehensive, integrated system for using a handheld telecommunication device to manage the logistics of medical device inventory throughout the lifecycle of the device.

### SUMMARY OF THE INVENTION

The present invention comprises a system and method of managing the logistics of trays and lots of surgical implants throughout the entire lifecycle of the device from the manufacturer's initial release until the instrument or implant is finally discharged by a medical service provider (i.e. disposing of an instrument or final expiration of the device). The system comprises software running on a central, secure server accessible by remote handheld telecommunication devices, such as smart phones, table computers, or the like. The software platform comprises several assemblies integrated into the system, the assemblies including: a database access layer assembly, a business rules assembly, an entity or business object assembly, a web service assembly, a database connection assembly, a presentation assembly for a handheld mobile device and a presentation assembly for a website and windows program. Additional functionality of the system comprises features such as a geographic map (i.e. Google® maps), an inventory list of medical assets such as trays and corresponding lots, an imaging assembly to read barcodes or other optic identifiers, an Food and Drug Administration (“FDA”) news feed assembly incorporating a live RSS feed for FDA releases about product defects or recalls, a web browser, and a notebook assembly to enter customized notes in free form text. The handheld device can access the software through a secure remote connection.

Users of the system are organizations that supply medical assets, such as medical device manufacturers, and in most cases each user has multiple representatives, each requiring remote access to the system from a unique handheld mobile device. A user must contact the administrator to obtain access credentials for the system. Upon initial activation of a user account with such credentials, the user creates a user profile by entering data pertaining to the user's representatives, existing medical asset inventory, and other relevant information.

During the life cycle of a tray, data about the tray and its corresponding lots is enter into the software system upon shipment by the manufacturer, and the software tracks the location of the shipment to its delivery destination, such as a hospital, medical facility, or other end destination. When the shipment reaches its end destination, an on-site representative of the user confirms the receipt location, thus logging the precise location of each tray by using the mapping assembly of the software. When a medical service provider discharges a lot, such as by implanting a device into a patient, the representative is on location to verify and log depletion of that particular lot by manually inputting appropriate data or by using the imaging assembly to scan the lot's optic identifier. Upon discharge of a lot, the software uses the inventory assembly to automatically send a message to the manufacturer to order corresponding replacement medical assets.

By accessing the inventory assembly, the representative obtains data about all medical assets in the user's profile, the location of the medical assets, and data about the medical assets, such as dates, times, the name of medical facilities, mailing address of the location, and other customized data. The FDA assembly uses a live RSS to provide the representative with hourly FDA warnings or recalls for specific lots. Thus, the representative, who is typically in the operating room during implant procedures, can prevent medical procedures that use devices for which the FDA has revoked approval.

The user application software additionally comprises a web browser so that the representative is not required to exit the software application to access the web browser running separately on the handheld device. The notebook assembly allows the representative to enter free form notes for any reason.

### BRIEF DESCRIPTION OF THE DRAWINGS

FIG. 1 is a schematic diagram showing one embodiment of the connectivity of the system's hardware components.

FIG. 2 is a diagram showing an alternate embodiment of the connectivity of the system's hardware components.

FIG. 3 is a diagram showing an alternate embodiment of the connectivity of the system's hardware components.

FIG. 4 is a schematic block diagram of the controller, processor, memory device, database, and computing device in one embodiment of the present invention.

FIG. 5 is a diagram showing one embodiment of the network connectivity of the system's servers.

FIG. 6 shows an alternate embodiment of the connectivity of the system's hardware, where at least a portion of the hardware is deployed in a cloud-based environment.

FIG. 7 shows a typical screen shot of the web application.

FIG. 8 is a diagram illustrating a typical software suite architecture used in the tracking system.

FIG. 9 is a diagram showing the interrelation of the software application's functionality modules.

FIG. 10 is a flow chart showing the steps of using the system to schedule a case.

FIG. 11 is a flow chart showing the steps of using the system to prepare a requisition form during a case.

FIG. 12 is a diagram showing the work flow in one embodiment of the loaner management module.

FIG. 13 is a diagram showing one embodiment of the loaner request submission process.

FIG. 14 is a diagram showing one embodiment of the loaner request submission process.

FIG. 15 is a diagram showing one embodiment of the steps of using the warehouse module during the sterilization and reconciliation process in the warehouse.

FIG. 16 is a diagram showing the steps of operating the system for logistics management.

### DETAILED DESCRIPTION

### I. Overview

With reference to the drawings, the invention will now be described with regard for the best mode and the preferred embodiment. In general, the invention is a system for and method of providing integrated logistics management of inventory, wherein the system is operable from a single application on a mobile telecommunication device, such as a smart phone, tablet computer, or the like. The system is capable of providing real-time tracking and logistics management of inventory throughout the lifecycle of each tracked object. The system is adaptable for use in a variety of industries, such as the medical, automotive, shipping, retail, or other industries. For the purposes of illustration, the system will be described herein in relation to logistics management of surgical implants. The embodiments disclosed herein are meant for illustration and not limitation of the invention. An ordinary practitioner will understand that it is possible to create many variations of the following embodiments without undue experimentation.

The system described herein is controlled by an administrator and used by sales representatives of medical device manufacturers. As used herein, the term “user” generally refers to the organizations that supply medical assets, such as medical device manufacturers. Other users of the system may include organizations such as a hospital or other health care provider, as will be obvious from the context. The term “representative” refers to a representative of the user, such as a sales representative of a medical device manufacturer.

In general, medical or surgical inventory comprises medical implantable devices and the tools and equipment needed to perform the surgery during which the medical devices are implanted. These medical assets are typically categorized into trays, and subcategorized into lots. Each tray is issued by the manufacturer with a unique alphanumeric identifier, or tag, and each lot inside the tray is further identified by a unique lot number. The lots are either the surgical implant devices or the specialized medical instruments needed to install such implants. As used herein, the term “medical assets” means trays or groups of trays and their corresponding lots.

### II. Components and Connectivity of the Tracking System

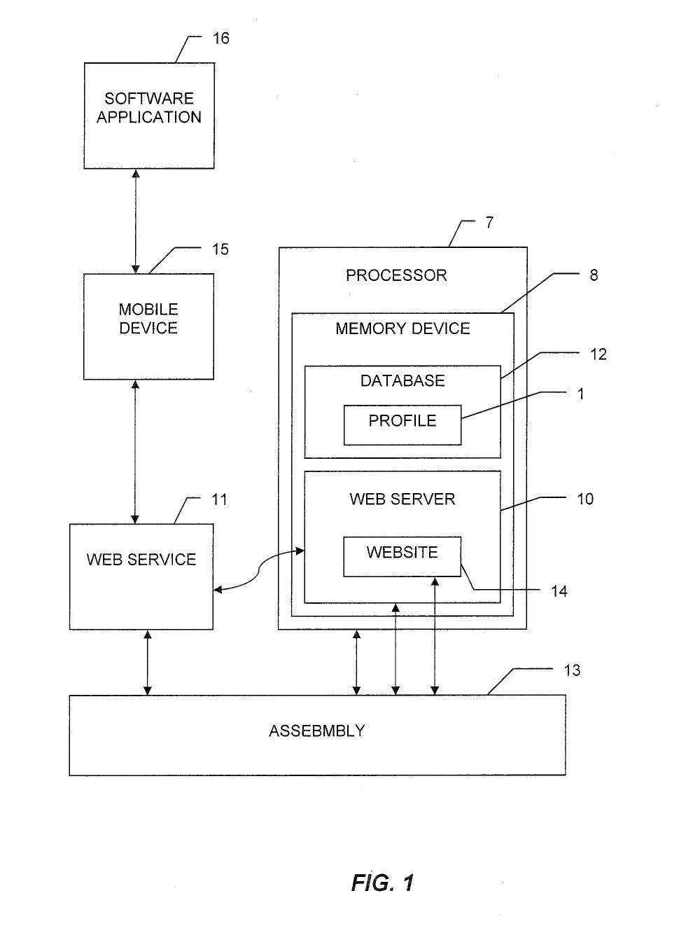

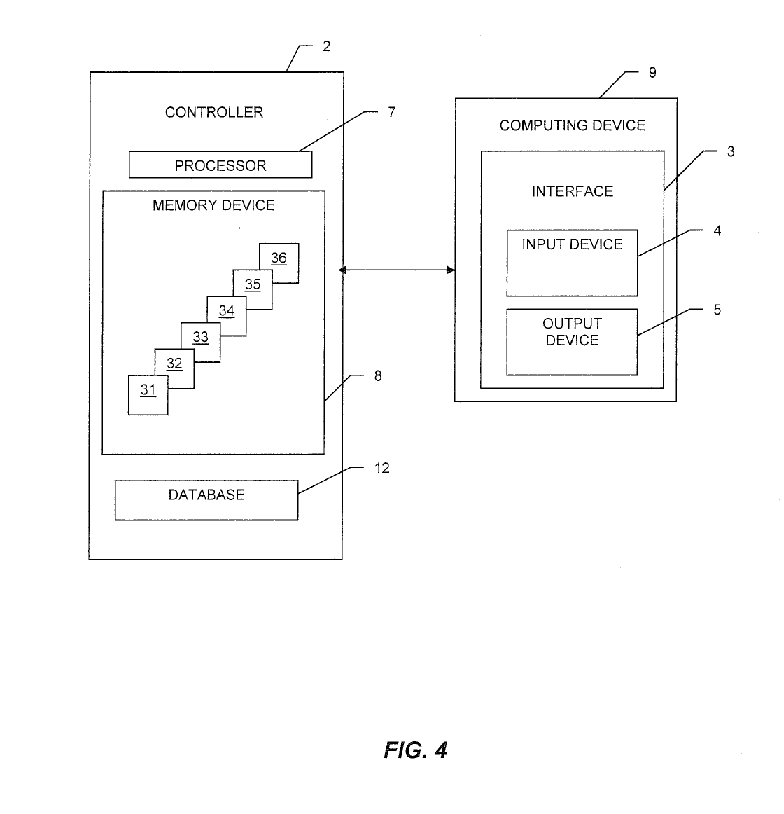

Referring to FIGS. 1-3, the most basic embodiment of the logistics management system generally comprises the following basic components: at least one processor 7 in communication with at least one memory device 8, a server 10, a communication service 11, a database 12, one or more assemblies 13 to facilitate communication between the database 12 and the communication service 11, a website 14, a consuming handheld mobile device 15 supporting a user software application 16. The processor 7 comprises one or more of a number of suitable computer processors capable of managing, processing, analyzing, storing, and retrieving the data within the system. One embodiment of the processor 7 is hardwired dedicated circuitry for performing the system functionality described herein, such as circuitry within a microprocessor, coprocessor, a controller, or various other hardwired circuitry. Alternately, the processor 7 is configured to execute computer readable code stored on the memory device 8, where the computer readable code contains instruction for executing the system functionality described below. The processor 7 could be a combination of hardwired dedicated circuitry and executable instructions. The processor 7 could be embodied within a controller 2 (see FIG. 4), embodied separately. The memory device 8 is operable to store computer readable program instructions designated for execution by the at least one processor 7. An example of the computer readable program instructions are the software suite modules discussed below. The memory device 8 includes one or more of volatile or nonvolatile memory components, several of which are known in the art. The memory device 8 is configured to store data, applications, modules, instructions or the like for enabling the system to execute the functionality described below in the various embodiments. The processor 7 and the memory device 8 are configured to interface with the peripheral components of the system described below.

In an example of one embodiment of the system, the server 10 is a web server, such as an SQL web server that supports the website 14, and the website 14 also communicates directly with the assembly 13. The communication service 11 is a means for communicating between the server 10 and the device 15 or other devices, such as computers or other communication entities, such as a variety of web services. The communication service 11 comprises one or more of an HTTP web request, an XML envelope, or TCP/IP protocol suite, or other suitable communication services. The database 12 is a database that is communicatively linked to the processor, and the database 12 is accessible via the network 17. The database 12 supports each user profile 1, which is a data profile containing data that relates to the user's representatives, existing inventory of medical asset, and other relevant information.

The assembly 13 is a library of compiled web services or other communication services 11. The user application 16 uses the communication service 11 to communicate with the database 12 and the assembly 13 via a network 17. The network 17 comprises one or more of a 3G or 4G network, LAN, WAN, broadband, the Internet, Wi-Fi, or other cellular or wireless networks, shown in FIG. 4. The wireless network 17 comprises one or more of these modes of wireless electronic communication.

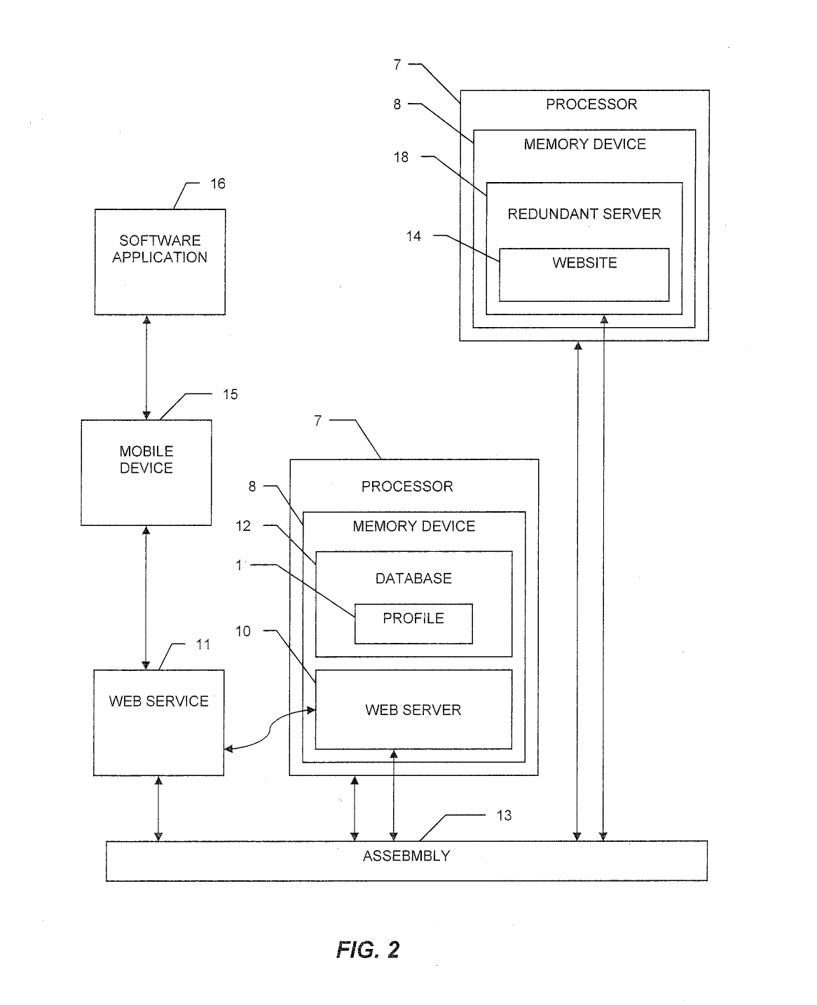

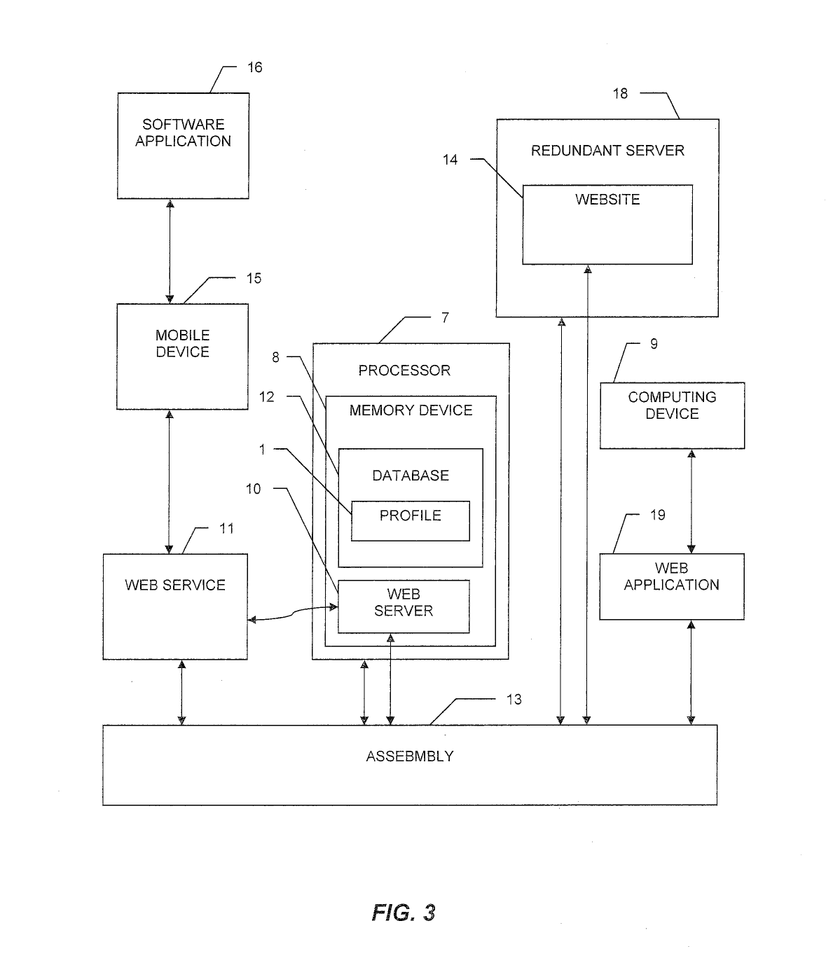

The server architecture of the system is a matter of design choice, and it may be advantageous in additional embodiments to provide one or more ancillary or redundant servers 18 to promote system reliability (see FIGS. 2, 3 & 5). In one embodiment, the servers 10, 18 are secured with digital security that complies with at least the minimum digital security standards set forth in the Health Insurance Portability and Accountability Act (“HIPPA”). The communication service 11 is comprised of one or more application programming interfaces (“API”), many suitable forms of which are commercially available or can be created without undue experimentation. In FIGS. 1-3, the mobile device 15 is one or more of a variety of handheld telecommunication devices, such as smart phones, tablet computers, or the like. The mobile device 15 comprises a GPS locator, as will be described in more detail below. The user software application 16 is a piece of software capable of being downloaded onto the mobile device 15 via the wireless network 17. The user software application 16 interfaces with the mobile device's 15 available features, such as the GPS geo-location feature, camera, video recorder, electronic map such as Google® maps, optical scanner, or other features. In another embodiment, the software application 16 comprises a web browser so that the user does not have to exit the software application 16 to access the web browser on the mobile device 15. The software application 16 also comprises a notebook, enabling the user to write customized notes about the medical assets or surrounding conditions, and associate such notes with a specific medical asset for later recall by the user.

As shown in FIG. 3, another embodiment of the system comprises a web application 19, such as a PC application or other application available via the world wide web, Internet, or other WAN, from which the user's manages its account and user profile 1 from a remote computing device 9, as described below. As shown in FIG. 4, the computing device 9 is any device that generally comprises a user interface 3, an input device 4, and an output device 5. The user interface 3 is operable to graphically illustrate output received from the system, and more specifically, from the processor 7. The user interface 3 can include an input device 4, such as a keyboard, mouse, touch screen, control buttons, voice activated commands, or other devices through which the user can input data and commands into the system. The user interface 3 can further comprise an output device 5, such as speakers, alarms, LED displays, a graphic screen having an LCD, plasma, touch screen, or other type of graphic or output display. In most embodiments, the remote computing device 9 is one or more of a desktop computer, a computing terminal, a mobile communication device, a workstation, or other device or terminal configured to communicate with the web application.

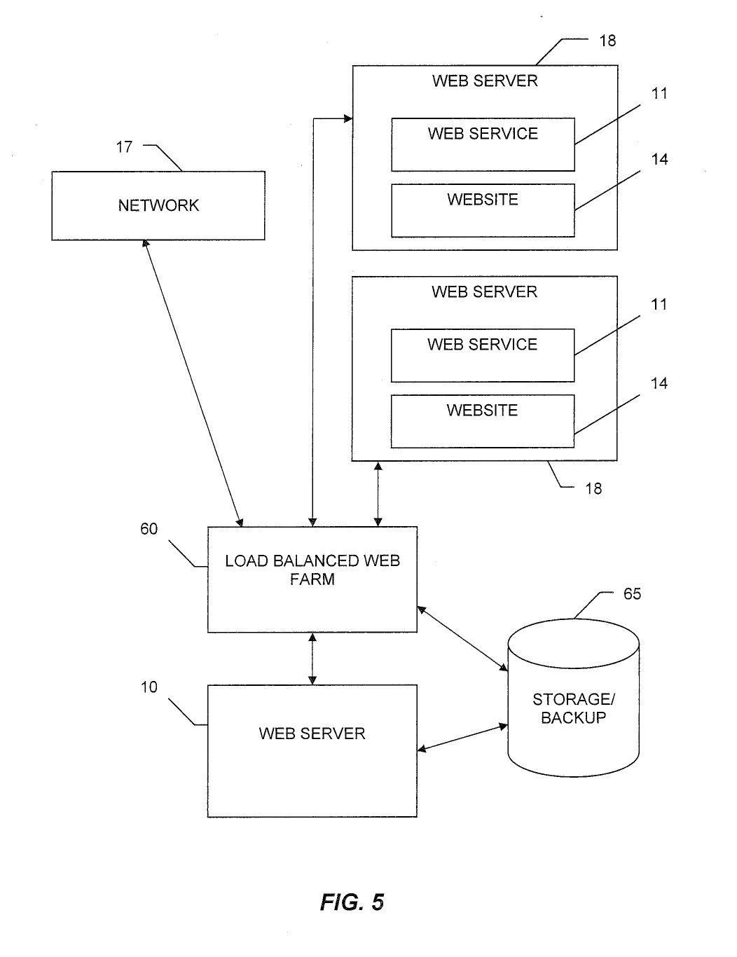

In another embodiment, shown in FIG. 5, the system comprises a load balanced web farm 60 supporting multiple servers 10, 45 in addition to support backup features 65, such as redundant servers and databases. The architecture of the web farm 60 is a matter of design choice and will vary by application. However, design of the web farm 60 design will not require undue experimentation of the tracking system.

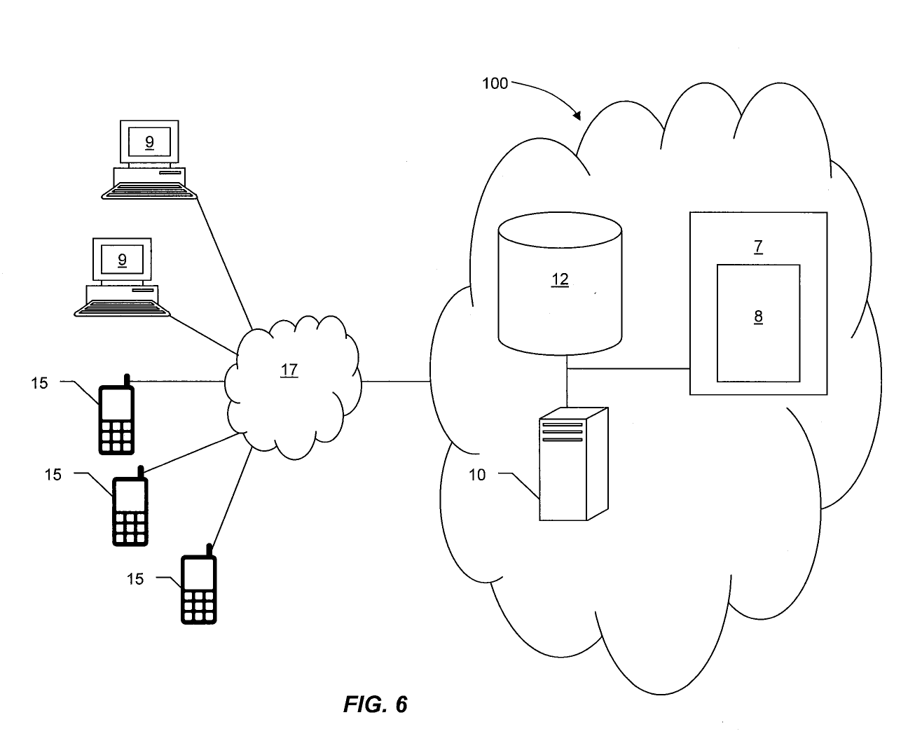

In another embodiment of the system, shown in FIG. 6, the system is deployed in a remote environment in the cloud 100. In this embodiment, the processor 7, memory 8, server 10, database 12, and other components are deployed in a cloud environment, which is accessible by the computing device 9 and the mobile device 15.

### III. Software Application

The system for and method of logistics management is driven by the software application 16 comprised of several assemblies 13, which are libraries, or groupings of programming code, that fulfill the following roles for the system to function: a database access layer assembly, a business rules assembly, an entity or business object assembly, a web service assembly, a database connection assembly, a presentation assembly for a handheld device, and a presentation assembly for a website and windows program.

III.A. Assemblies.

(a) Database Access Layer Assembly.

This assembly abstracts from the other assemblies 13 all activity related to the database 12, such as the activity of retrieving or saving data. The database access layer assembly makes itself available for communication with other assemblies without being directly tied to them. This process is also known as encapsulation.

(b) Business Rules Assembly.

The business rules assembly facilitates all related logical operations necessary to provide functionality to the system. Examples of this functionality include user account management, inventory management, execution of available features of the mobile device 15 or software applications, or other functions allowed by the system. The business rules assembly also uses an encapsulation process, and the assembly is configured for use in any direction in the logic flow without exposing anything internal to an external assembly 13 communicating with this assembly 13.

(c) Entity Object Assembly.

The entity object assembly provides a programming model based data schema. This assembly creates, manages, processes, and destroys all data that conceptually correlates to the real-space facts and information about items or objects of interest. For example, the entity object assembly is configured to create, manage, process, or destroy data relating to trays, companies, delivery status parameters, location, or lifecycle of a tray or its associated lots.

(d) Web Service Assembly.

The web service assembly enables a device to process programming logic required for account validation, inventory management, and business rule processing by placing the applicable code assemblies on a remote server 10, 45. The mobile device 15 then invokes logic embedded in the remote assemblies 13 without having to house or store any of the programming logic on the device itself. The web service assembly can also be accessed by the associated user application 16 the same way.

(e) Database Connection Assembly.

The database connection assembly 13 encapsulates all required logic to access the inventory of medical assets, account, and user database by providing a mechanism that enables the communication service 11, when invoked, to access the database connection assembly 13 and, in turn, retrieve the requested data and pass it back through the communication service 11 to the device 15 or user application 16.

(f) Presentation Module Assembly.

The presentation module assembly related to the mobile device 15 comprises all programming logic that is required to be installed on the mobile device 15 itself, which includes rendering logic for buttons, tables, text, and the navigation features permitting uninhibited navigation through the user application 16.

(g) Presentation Assembly.

The presentation assembly relates to the assemblies 13 and programming code responsible for the display of data, buttons, text and tables on the associated user application 16.

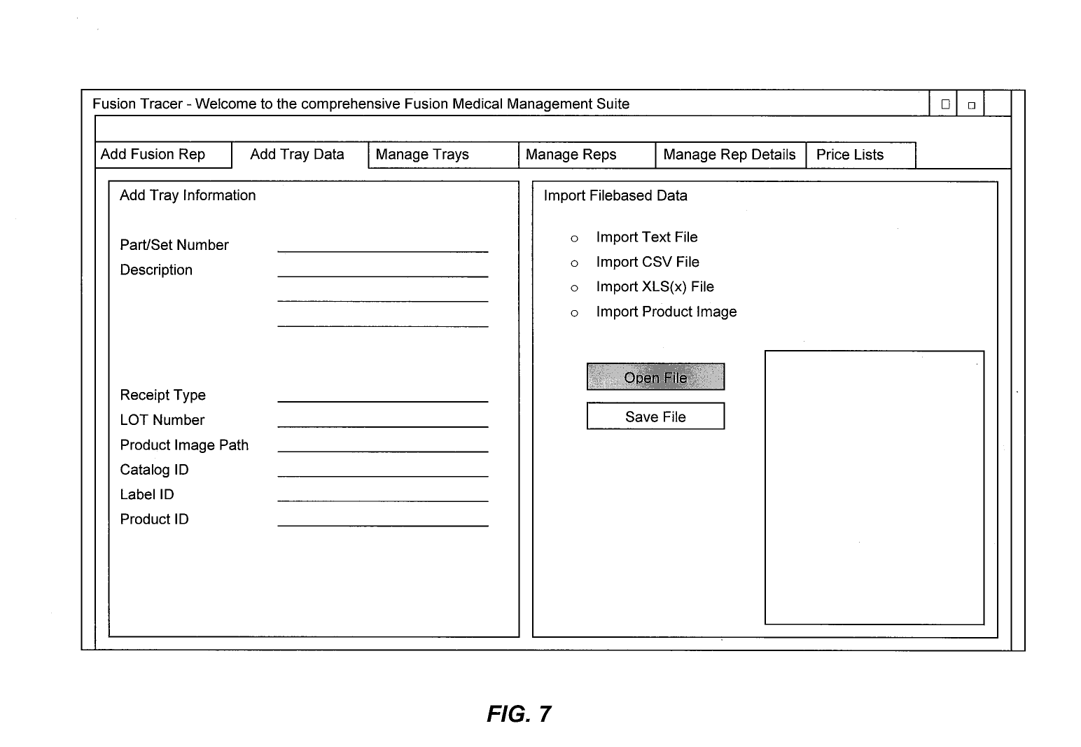

By way of further examples, FIG. 7 shows the typical screenshot of the web application 19, which would typically be operated by the administrator. The web application 19 includes features for managing sales teams and trays, such as by adding and managing representatives, adding and managing tray data, updating price lists. The web application 19 may also comprise fields for entering data, such as tray or lot information or other fields. The mobile device 15 uses the communication service 11 to communicate with the server 10. These features are comprised within a software suite 30, as described below.

### IV. Software Suite

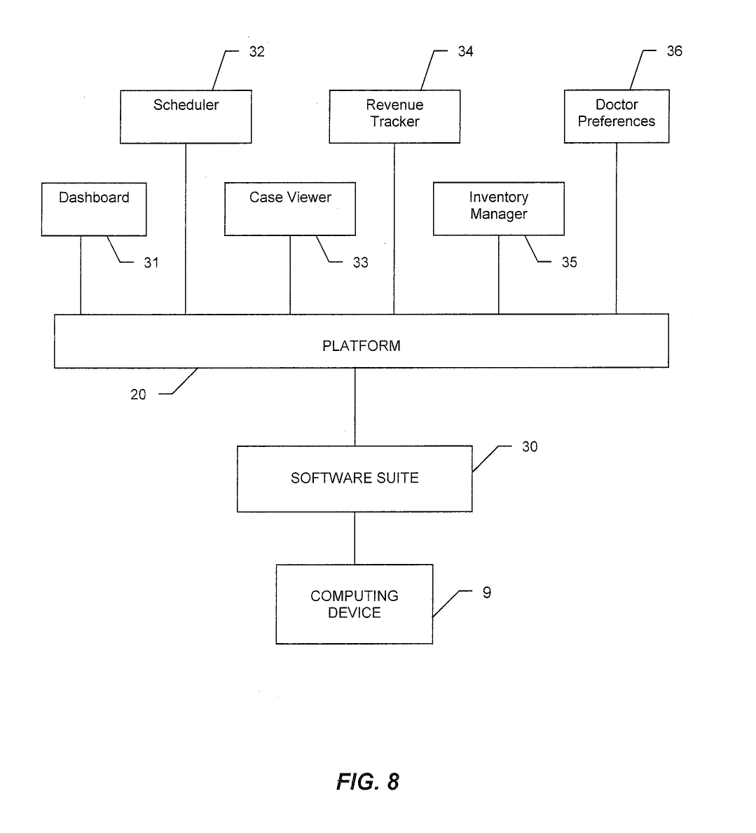

Referring to FIG. 8, the system comprises a software suite 30 that forms the interface between the user and the system, enabling the user to manage the flow of process of the system and interact with data stores. The software suite 30 is a package of software modules that comprise the functionality described below. In most embodiments, but not all, the software suite 30 is deployed in the form of a web suite or an Internet suite as desired. The features and functionality of the software suite 30 are deployed in modules, which are added or adjusted to accommodate a variety of users, needs, or applications of the tracking system. The user uses the software suite 30 to access all data stored in the user's profile 1, which enables the user to have a centralized management system for all of its representatives, facilities, providers, and patients. In typical embodiments, the software suite 30 comprises modules such as a dashboard 31, scheduler 32, case viewer 33, revenue tracker 34, inventory manager 35, and doctor preferences 36, to name a few. The dashboard 31 is the graphical interface between the user and the system, and the dashboard 31 displays visual indicators for access to the other features and processes. For example, the user uses the dashboard 31 to access the system during the user authentication process and to set up the user account and profile 1.

The scheduler 32 enables the user to send and receive scheduling data and information to and from the database 12. The user inputs, retrieves, or edits information relating to schedules for medical asset delivery, cases, representatives, or other such events, people, and processes. The case viewer 33 enables the user to access all information relating to a particular case, such information including data relating to the representative, facility, provider, patient, date of the case, the medical assets to be used, and other data relevant to the particular application of the tracking system. The case viewer 33 enables the user to have access to all cases within the user's profile 1, and to display certain data during a certain time period. For example, the user can retrieve and view all revenue received from facility X on day Y. A wide variety of other reporting parameters can also be selected.

The revenue tracker 34 enables the user to access, review, and store all electronic documentation and data relating to payments for and revenue from the medical assets used in the cases. For example, such electronic documents include electronic versions of one or more of purchase orders, requisitions, receipts, financial documents, or the like. Revenue data includes one or more of sales price, discounts, rebates, financial and lending data, and other such information. The revenue data is sorted into categories correlating to one or more of the representative, facility, case, doctor, a specific medical asset, or the like. Revenue data is tracked and reported for specific time intervals in connection with other data selection criteria. For example, the user could use the revenue tracker 34 to retrieve and view the revenue generated by representative X during month Y. In one embodiment, the revenue tracker 34 is configured to interface with the user's accounting software so that the user's accounting books are automatically updated with revenue information.

The inventory manager 35 enables the user to manage all medical asset inventory included within the user's profile 1. For example, the user accesses, reviews, and edits data relating to one or more representatives, the medical assets currently at one facility, medical assets allocated to one or more cases, and all data about the medical assets, such as the manufacturer, the manufacturing date and batch number, the delivery date, expiration date, quantity, contents, or other such pertinent information. The inventory manager 35 enables the user to perform a “radius search,” which will return to the user information about all medical assets within a specified radius of a certain geographic location. Thus, the user is able to readily identify all medical assets within a certain medical facility, zip code, town, or other geographic area.

The doctor's preferences 36 feature enables the user to access, edit, and view information relating to preferences by certain doctors and other medical providers with whom the user's representatives typically interact. For example, the user uses the doctor's preferences 36 feature to log information such as the weekdays on which a certain medical provider prefers to perform certain types of cases, or that the provider prefers one brand of implant device over other brands. Any such unique preferences by the doctor or medical provider are recorded by the representative and stored in the system on the database 12 for future reference by the administrator, user, or other representatives.

The software suite 30 comprises additional features or functionality, depending on the needs of the user or other determinative considerations.

### V. Application Functionality and Platform

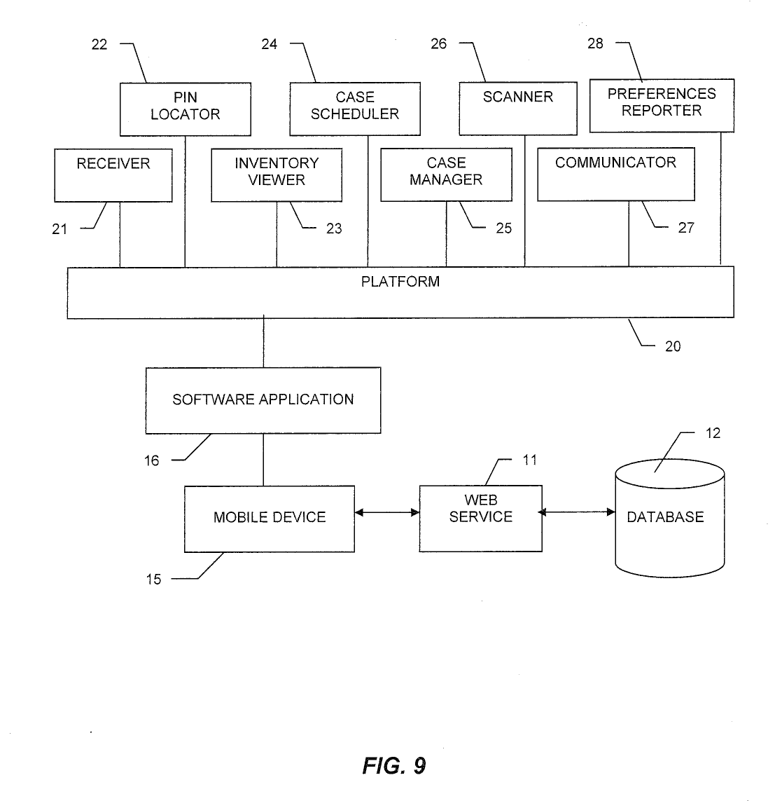

Referring to FIG. 9, the software application 16 comprises a platform 20 incorporating multiple functionality modules that are selected and arranged as appropriate for the particular purposes of the tracking system. An exemplary list of modules includes a receiver 21, pin locator 22, inventory viewer 23, case scheduling module 24, case manager 25, optical scanner 26, communicator 27, and preferences reporter 28, among others. The representative uses the mobile device 15 to activate the software application 16 to access each of these modules and perform the functionality delivered by each module, as described below.

The receiver 21 is an onboard medical asset input utility in the software application 16 on the mobile device 15 that functions as a graphical interface for data entry into the database 12, as well as data retrieval from the database 12. The representative sends and receives to and from the database any data relating to medical assets, including, without limitation, information pertaining to specific trays or lots, biologistics, delivery information, quantity, expiration date, geographic or facility data, or any other stored data relating to the user, the profile 1, medical assets, doctor, patient, facility, or the like. The representative also uses the receiver 21 to allocate lots into a tray, if so required, by capturing part number, lot number, part description, expiration date, and quantity within the context of the distributor. This enables the distributor to add unforeseen or newly discovered medical assets into the profile 1 aside from possibly receiving EDI data medical assets transfers into the user's profile 1. In addition, the receiver 21 enables immediate GPS based location pinning of the entered data at the location where the input occurs, as described below.

The pin locator 22 is a module used to pinpoint a geographic location. For example, the pin locator 22 is configured to pinpoint the geographic location of the mobile device 15, and therefore the representative. Additionally, the pin locator 22 is used to “pin” the geographic location of medical assets by using the GPS locator in the mobile device 15. The medical assets are “pinned” when the user uses the pin locator 22 to record and store the geographic data associated with the current geographic location of the medical assets. The pin locator 22 is configured to report geographical locations in a variety of forms, such as by recording and storing one or more of street addresses, the geographic coordinate system (i.e. latitude and longitude), or other descriptors of geographic location. For example, the latitude and longitude readings returned from the GPS feature in the mobile device 15 can be converted to a street address and displayed on an electronic or online map, such as Google® maps. As the physical location of the medical assets changes with time, the user updates the recorded location by re-pinning each medical asset as needed. Later, when the specific location of a medical asset is needed, the user uses the inventory viewer 23 module to determine the last pinned location of the inventory, as described below.

The inventory viewer 23 module enables the user to view the current medical assets in the user's profile 1, including a view of the current tray and lot numbers and any other parameters stored in the database 12 in association with the medical assets. Quantity, status, availability, and location may be such parameters, if so desired by the user and stored in the database 12 in the user's profile 1. The inventory viewer 23 also permits the user to edit the inventory listings, such as by deleting mobile device 15 entered data, entering notes pertaining to medical assets data, or editing other parameters.

In one embodiment, the inventory viewer 23 provides the capability of immediately searching and reporting medical assets in a given location, such as searching by the radius in miles and name of the device, implant, or tray. This example is called a radius search. In another embodiment, the inventory viewer 23 provides satellite and street level views of the location of current medical assets, along with a list of parts and lots that are potentially part of a tray, device, or implant. In another embodiment of the inventory viewer 23, the main screen also provides for searching not only the tray, device, or implant by name, but also allows the user or distributor the option to perform an FDA regulation-based search by actual part or lot numbers, and the inventory viewer 23 displays the location of any medical assets found.

In another embodiment, the inventory viewer 23 also provides a tracking means, which is a means for tracking the location as a function of time for each medical asset throughout its entire lifecycle. For instruments, the lifecycle begins at the time of the initial shipment by the manufacturer until the time that the instrument lot is depleted, such as by completing a surgery or final disposal of the instrument lot or tray by a healthcare provider. For implant devices, the lifecycle begins at the time of the initial shipment by the manufacturer and continues until the time that the device expires, which may be several days, weeks, or years after the device is implanted into the patient. When a tray or lot is depleted from the medical asset inventory, such as by discarding the lot or implanting it into a patient, the representative updates the corresponding medical asset data, and the system will automatically notify the manufacturer that a replacement tray or lot is needed and the location of where such medical assets are needed. The tracking means includes the functions of identifying, logging, and recording a tray number or a lot number, and recording the time and location of the tray or lot during the entire lifecycle. In another embodiment, the tracking means comprises tracking parameters carrying additional information about the medical assets, such as the surgery location, physician name, tray identification, a picture of the facility, or other information as desired. The system enables the tracking function by connecting to the database 12, through the same communication service 11 that the mobile device 15 is remotely hydrating and utilizing.

In another embodiment of the system, the inventory viewer 23 further comprises a reporting function that provides a web based view for users or representatives to access the tray, lot, surgery data, or other tracking parameters in real-time as soon as data has been entered. This view, which is always associated with unique login credentials (e.g., for the representative), provides for a map view, a data view and detailed breakdown view pertaining to the data being logged on the remote handheld device. Such data could include location (GPS) data, detailed tray information, surgery data, or other tracking parameters the representative has entered. The system enables the reporting function by connecting to the database 12, through the same communication service 11, that the mobile device 15 is remotely hydrating and utilizing.

The case scheduling module 24 is a module that permits the user to schedule surgery procedures, or “cases,” in which medical assets are to be used or depleted. In the case scheduling module 24, the user enters or edits parameters relating to each case, thereby defining a set of case information data 51 for each case. The case information data 51 comprises a list of one or more medical assets that are required to perform and complete the case, as well as information corresponding to one or more of the date and time that the case is to be performed, the type of health care procedure that is to be performed, the name and location of the health care facility where the case is to be performed, nature of the medical procedure to be performed, the name of the medical provider that is to perform the case, an identification of the patient that will undergo the medical procedure, the room number for the procedure, and any other relevant or desired information. The case scheduling module 24 also enables the user to modify or edit the case information data 51 as needed or desired.

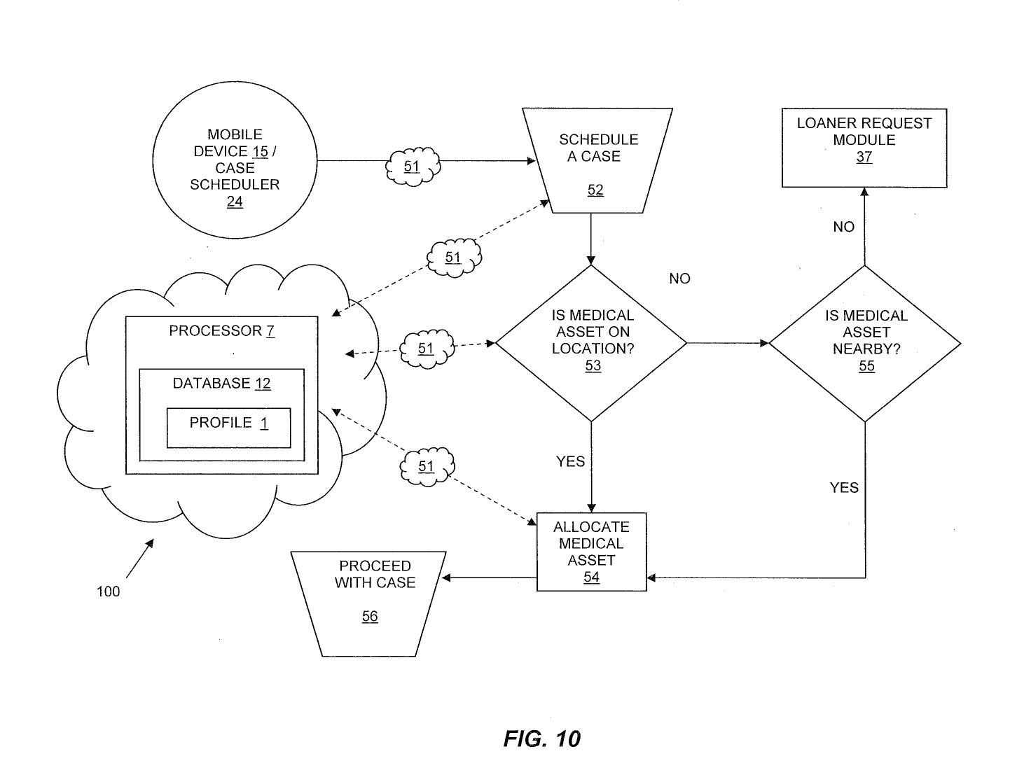

More specifically, referring to FIG. 10, a case is initiated by the health care provider, such as a surgeon or other health care practitioner. The provider provides the case information data 51 to the representative. The representative uses the mobile device 15 to invoke the software application 16, thereby obtaining access to the case scheduling module 24. The case scheduling module 24 provides an interface through which the representative performs the steps of scheduling the case 52 by entering the case information data 51 into the database 12 via the assemblies 13. The representative then accesses the user's medical asset inventory list in the user profile 1 to make a determination as to whether the medical assets requested for the case are currently available on-location 53 at the health care facility. If the requested medical assets are currently available on-location, the representative uses the application 16 to perform the step of allocating the inventory 54 for the scheduled case. The medical assets are then designated in the user's profile 1 as allocated and unavailable for other cases at the time of the scheduled case.

If the requested medical assets are not currently available on-location at the health care facility, the representative accesses the user's medical asset inventory list to perform the step of determining whether the tray is nearby 55 and available at an off-site location, such as a nearby health care facility. If the tray is available at an off-site location where it can be timely delivered for the scheduled case, the representative uses the application 16 to perform the step of allocating the tray 54 for the scheduled case. This step is automatically performed by the processor 7. The tray is then designated in the user's profile 1 as allocated and unavailable for other cases at the time of the scheduled case. The representative then performs the step of proceeding with the case 56 at the time that the case is scheduled to be performed.

If the required tray is not available in the user's medical asset inventory list, the representative uses the application 16 to access the loaner request module 37 to obtain the required medical assets for the scheduled case. The loaner module is discussed in detail below.

In most situations, each user has multiple representative, each of whom may have multiple open cases at any given moment. The processor 7 automatically collects the case information data 51 from each case of each of the user's representatives. The processor 7 aggregates this collected case information data 51 to construct a master schedule for each user as the user's representatives enter case information data 51 to populate the master schedule. Each representative has access to the user's master schedule so that case information data 51 and medical asset information are always available to each representative. The master schedule is updated in real-time so that the representative's view of the medical asset inventory and related information is always current and based on up-to-the-minute changes.

In another embodiment of the case scheduling module 24, after the representative receives the initial case information data 51 from the healthcare provider, the representative uses the mobile device 16 to invoke the case scheduling module 24 via the software application 16. Using the case scheduling module 24, the representative transmits the case information data 51 to the user profile 1 by using the case scheduling module 24 to execute a wireless transmission of the case information data 51 from the mobile device 15 to the database 12, thereby populating the profile 1 and master schedule. The user profile 1 comprises a list of available medical assets, which identifies a plurality of the medical assets in the user's profile 1. The processor 7 automatically performs the step of determining whether any of the medical assets in the list of available medical assets are responsive to the requested medical assets. If so, the processor 7 performs an automatic determination of whether the responsive medical assets are currently available at the location of the healthcare facility where the case will be performed 53. If this determination is positive, then the processor 7 automatically performs the step of allocating the responsive medical assets to the case being scheduled 54. The processor 7 then automatically sends a notification to the representative that the required medical assets have been located and allocated.

If the step of determining on-location asset availability 53 is negative, then the processor 7 automatically performs a search for medical assets identified in the list of available medical assets that are currently available from an off-site location. The processor 7 performs the step of determining whether the required medical assets are available from the list of available medical assets at the location nearby the healthcare facility where the case will be performed 55. It is preferable that the processor 7 is configured to calculate the shipping time from the current location of the off-site medical assets to the location where the case is to be performed. Off-site medical assets that cannot be timely delivered to the healthcare facility for the cases are designated by the processor 7 as unavailable for the case. If the processor 7 is able to locate off-site medical assets that meet the medical asset requirements for the case and can be timely delivered, then the processor 7 automatically performs the step of allocating the available medical assets to the case being scheduled 54, and the processor 7 sends a notification to the representative that the required medical assets have been located and allocated.

Once the representative has obtained the required medical assets, the scheduled case is performed by the provider. During the surgical procedure, the representative is on-location in the operating room logging the medical assets, by both trays and lots, used by the provider in performing the surgical procedure. The representative performs these steps by using the mobile device 15 to invoke the application 16, thereby obtaining access to the case manager 25.

The case manager 25 is a module used by the representative during a surgical procedure create, log, and manage case management data 60. Case management data 60 comprises information pertaining to the user's inventory levels, the lots used during the case, the tray and lot numbers for the used medical assets, the purging of those used lots from the user's inventory list, requisition information for depleted medical assets, billing information, and other such information related to the medical assets used during the case. The case manager 25 module displays input form fields and medical asset data corresponding to the scheduled case. The system then, through a method called in the web service assembly 13, stores in the database the tracking parameters pertaining to the case-specific tray or lot, as identified by the tray or lot number. This storage procedure, or precompiled structured query language statement, facilitates the normalized distribution of the case management data 60 in the data tables in the database 12 responsible for saving tray, surgery, lot data, tray data, or other tracking parameters or case management data 60. Thus, the case manager 25 gives the user immediate purge control over all medical assets used in the case, deleting any depleted stock out of the system in real-time.

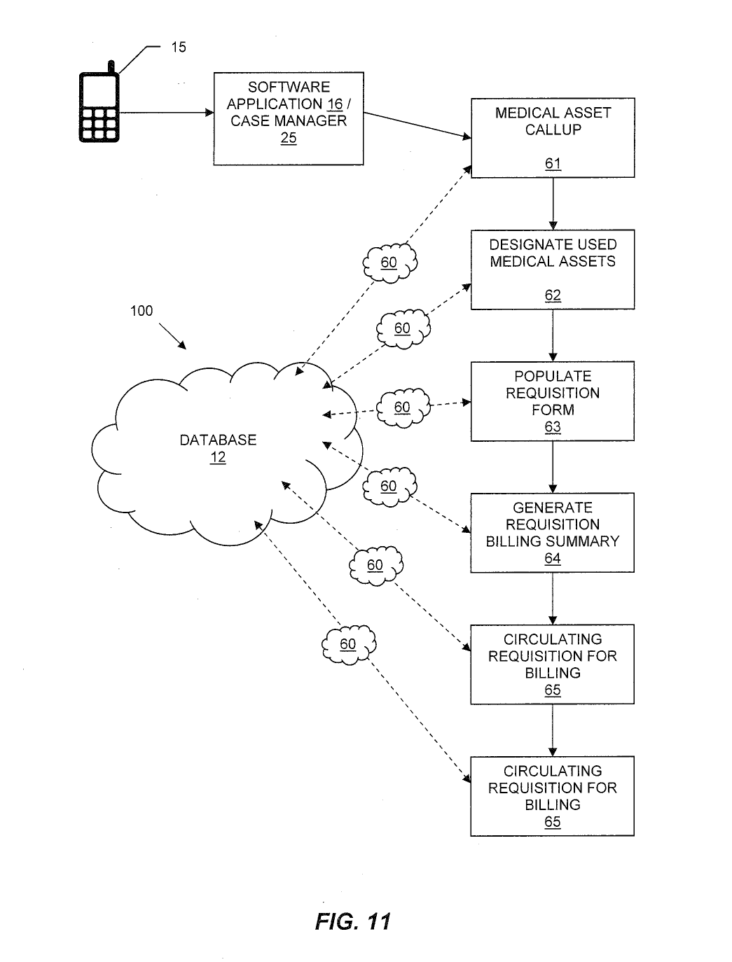

For example, referring to FIG. 11, at the beginning of the surgery, the provider will physically open a tray by removing the sterilization packaging from the tray. At that time, the representative uses the application 16 to access the case manager 25 to perform the step of calling up the specifically scheduled medical asset 61 from the user's medical asset inventory list, which is stored in the database, for management during the case. As the provider uses the lots during the procedure, the representative uses the case manager 25 for the step of designating used medical assets 62, such as used lots, accordingly. The representative repeats these steps for each tray and lot used by the provider during the case. At the end of the procedure, the representative has compiled an accurate set of case management data 60 listing of all medical assets used, both trays and lots. The representative then uses the requisition system as described below.

The case manager 25 also provides for an electronic requisition system for replenishing medical assets that were used or otherwise depleted from the profile 1 of medical asset inventory during the provider's performance of a case. In one embodiment, for example, the requisition system captures all parts, lots, prices, descriptions, and other relevant case management data 60 onto a template or custom requisition form that is submitted to the manufacturer allowing for immediate replenishment of tray or device lots. In other words, the system automatically performs the step of automatically populating an electronic requisition form 63 with case management data captured in real-time during the surgical procedure.

The representative also uses the case manager 25 to create requisitions for billing purposes. This requisition form is a re-order form to replenish exhausted or depleted medical assets. For example, when a lot is used in a surgery, the representative designates the lot as used, as described above. When the medical procedure of the case is complete, the system automatically performs the step of generating an electronic requisition billing summary 64 of all medical assets used in the case. The representative then performs the step of electronically circulating the requisition billing summary 65 to the relevant billing parties, such as the healthcare facility, the provider, the manufacturer, and the like. For example, the requisition form electronically circulated to the manufacturer for replenishment of medical assets, as described above, and also sent to the provider or health care facility to process billing, accounting, and payment procedures. This real-time processing of medical assets enables immediate and accurate billing and replenishment of inventory, without the representative relying on hindsight to recall the medical assets used and the appropriate billing information. This prompt preparation and distribution of requisition forms enables prompt and accurate billing and replenishment of medical assets, thereby avoiding subsequent disputes between the representatives, providers, users, and heath care facilities.

In some instances, completion and processing of a requisition form requires the step of obtaining authorization signatures (step not shown in figures) from the representative, provider, health care facility, or other party. Therefore, one embodiment of the case manager 25 comprises functionality in which the requisition is automatically prepared for signature when the representative designates medical assets as used, exhausted, depleted, or expired in the course of a case. When such medical assets are removed from the representative's inventory listing, the case manager 25 automatically hydrates an electronic requisition form with the data necessary to reorder such depleted medical assets. The representative, doctor, or other authorizing party then uses the touch-activated screen on the device 15 to sign the requisition form via a “signature capture.” The signature capture allows the user to use his or her finger to trace his or her signature onto the touch screen. The graphics capability in the touch screen framework capture a line-form digital image of the signature in a common format, such a JPEG, TIFF, bitmap, or the like. The signature capture then converts the signature image to a binary code capable of being transmitted over the Internet as a data file. This data is transmitted to the server 10 where the data is reassemble on the server 10 and inserted into the signature line of a document. For example, the reassembled signature could be inserted into the signature line of the “requisition image” of the completed and fully hydrated requisition form permanently tagged with the image of the requisition form with the image of the signature. The requisition image is then made available for access via the Internet by the device 15, software suite 30, a web browser, or other means. This process will appear as a real-time action to the signatory. The case manager 25 is also configured to email a copy of the requisition image to the representative for subsequent review and processing. The final requisition form is electronically transferred to the representative for distribution to the healthcare facility or manufacturer.

The optical scanner 26 module comprises an imaging assembly capable of reading and logging data contained in an optic or electronic identifier, such as a bar code or an RFID tag. The optical scanner 26 module serves as a backup option to identify medical assets. In many cases, the manufacturer will affix a barcode to the medical assets as a means of optical identification. The barcode contains unique identifying information about the tray or other medical assets. In one embodiment of the present system, the system incorporates a mobile device 15 that comprises a camera capable of capturing the barcode image, and software within the mobile device 15 will process the image to extract the numerical value of the barcode, thereby identifying the medical assets. In more advanced embodiments of this system, the camera and mobile device 15 are capable of capturing and processing multiple barcodes in one image.

In another embodiment of the present system, the optical scanner 26 comprises bluetooth laser reader, which is detached from the mobile device 15, but offers the user the most generic barcode reading capability in the industry. The laser reader permits the user to decide which entry field within the receiver 21 or case manager 25 is targeted by the bluetooth laser. This, in turn, gives the user the ability to decide if a part or lot or other barcode is within the context of the selected field.

The communicator 27 module provides a chat client available to the user and representatives registered to the user's profile 1. Thus, the communicator 27 provides an “intra-company” or “intra-profile” communication forum having features such as SMS text message options, thread-discussion forums searchable by topic, and a stored data log that retains all communications in a database. These and other such features of the communicator 27 are selected and arranged as desired for a particular purpose without falling outside the scope of the present system.

The preferences reporter 28 is a knowledge base available within the mobile device 15. The preferences reporter 28 is configured to receive, store, and recall on-demand a variety of information such as the physician's name, place of business, affiliation with healthcare providers, preferred medical assets, preferred style and manner of surgery preparation, or the like. The information stored in the preferences reporter 28 can be retrieved and displayed in a scrollable format.

In another embodiment, the application 16 comprises a news feed that receives real-time industry based news releases, such as releases from regulatory agencies. For example, the news feed is configured for receiving news releases from the Food and Drug Administration regarding warnings, alerts, or recalls corresponding to certain medical assets.

These and other modules are selected and arranged as needed to suit certain user profiles 1 or applications of the medical asset tracking system, which is not limited to any particular selection or arrangement of these modules.

### VI. Loaner Management Module

VI.A. Overview

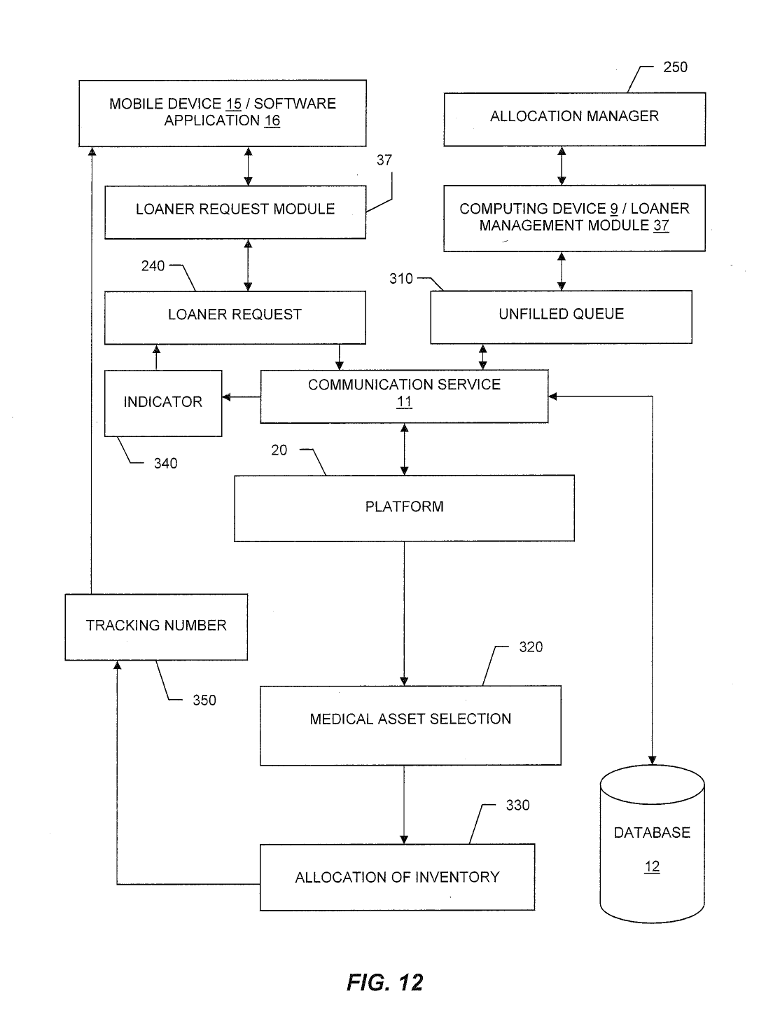

Referring to FIG. 12, in one embodiment of the tracking system, the software application 16 further comprises a loaner request module 37, which is deployed either as a separate module in the software application 16 or as a feature of the case scheduling module 24. The loaner request module 37 provides functionality that enables a sales representative to acquire access to medical assets that are not available in the user's profile 1. The medical assets may be unavailable because the user does not carry that type of inventory, or it may be unavailable because all of the user's medical assets of the required type are allocated to other cases at the time the representative will need the medical assets for the case being scheduled. To support the loaner request module 37, the tracking system is modified so that the user can edit its user profile 1 by designating one or more other medical asset manufacturers with which the user would like to be associated. These designated manufacturers are available to the user to receive loaner requests 240 submitted by the user's sales representatives when the user does not have the required medical assets in stock, at the required location, or otherwise readily available. The user populates a loaner inventory list using medical asset information made available by its designated manufacturers.

A loaner request 240 comprises a list, or designated set, of generalized tray types that are needed for the scheduled case and are not otherwise available in the user's existing medical asset inventory. The loaner request 240 includes a set of specific loaner tray serial numbers that may be referred to as a “unit” or “kit.” Depending on the manufacturer, kits may be built using one of two assembly methods: (1) Ad-hoc and (2) Kit Definition. The ad-hoc method is the method by which a set of trays is bundled to form a kit without regard to the tray's individual serial number. Generally, ad-hoc is where a family of tray types is bundled at the time of allocating trays to form the kit. The kit definition method is the method by which specific tray serial numbers are always bundled collectively in order to form the kit. In this model, special kit number characteristics are stored on each tray record in the tracking system to enable tracking all trays belonging to a single kit. At the time of tray allocation, the allocation manager views this kit characteristic to ensure that only trays of the same kit are being allocated.

Generally, the representatives uses the loaner request module 37 to initiate any needed loaner requests 240 at the time of scheduling a case. The representative uses the mobile device 15 to invoke the software application 16, thereby obtaining access to either the loaner request module 37 or the loaner request module 37 feature within the case scheduling module 24, depending on how the system is designed.

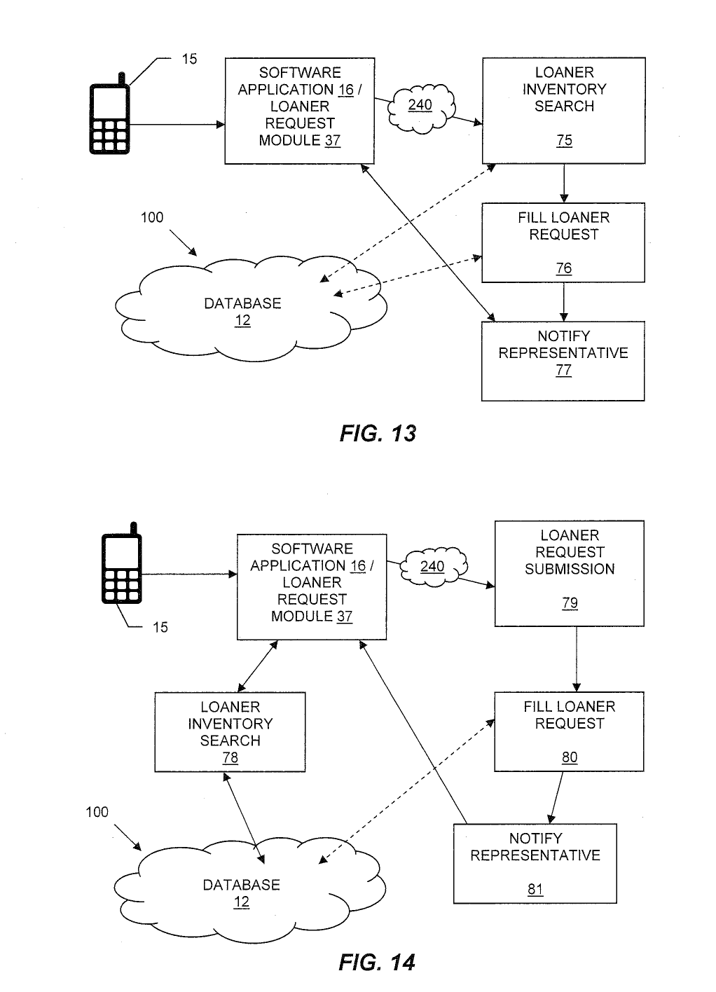

In one embodiment of the loaner request module 37, shown in FIG. 13, the representative uses the loaner request module 37 to communicate the loaner request 240 to the user via the system. After the user receives the loaner request 240, the user searches its available loaner inventory list 75 to determine whether the required kit is currently available from another manufacturer. After locating the required medical asset in the loaner inventory list, the user fills the loaner request 76 by sending a notification to the representative that the required kit has been located and that it will be sent to the representative, as discussed in more detail below. The loaner kits then appear in the representative's loaner request module 37 interface under the delivery designations discussed below.

In another embodiment of the loaner module process, shown in FIG. 14, the representative uses the loaner request module 37 to access the user's available loaner inventory list to perform an inventory search 78 to determine whether the required kit is currently available from another manufacturer. After locating the required medical asset in the loaner inventory list, the representative submits the loaner request to the user 79 via the system. The system then notifies the user or its designated manufacturer that the loaner request 240 is ready to be filled and shipped, as discussed below. The user then arranges for the loaner request 240 to be filled 80, and notifies the user that the request 240 is filled 81. In either of these allocation embodiments, the representative or the user can perform a radius search as described above to locate available loaner inventory.

VI.B. Loaner Request Lifecycle

Referring again to FIG. 12, the life-cycle of a loaner request 240 consists of four stages designated a visual indicator 340 identifying the four indications: (1) unfilled, (2) filled, (3) shipped, and (4) cancelled. When the loaner request 240 is initially saved, it enters the system under the default status of “unfilled.” After the user or designated manufacturer hard allocates 330 a kit to the loaner request 240, the loaner request's 240 status in the system changes to “filled.” When the kit is shipped by the manufacturer, the status changes to “shipped” and is assigned a tracking number 350. The representative may choose at anytime in the life-cycle other than “shipped” to cancel the loaner request 240. A cancelled loaner request 240 releases any hard allocations associated with it.

VI.C. Tray Allocation Process

In a common embodiment of the loaner system, the software suite 30 further comprises a loaner management module 38. The manual tray allocation process begins with the user's allocation manager 250 using the computing device 9 to invoke the web application 19, thereby obtaining access to the software suite 30 and the loaner management module 38. The allocation manager 250 then uses the loaner manager module 37 to select a loaner request 240 from the unfilled queue 310. Based on the selection of a loaner request 240, a list of requested tray types in the loaner request 240 is automatically obtained. The manager 250 compares this list of tray types against the loaner inventory database for all tray numbers belonging to the tray types identified. The tray status, or availability, is then indicated in the loaner management module 38 by a visual indicator 340, such as a light, icon, distinct font, or the like. The manager 250 then “hard-allocates” 330 available trays to the loaner request 240. When a tray type is unavailable, the manager 250 uses the loaner management module 38 to send an “unavailable” indication to the sales representative via the loaner management module 38 in the sales representative's 50 case scheduling module 24. The representative can either select an alternate tray type, cancel the loaner request 240, or remove the unavailable tray from the loaner request 240, allowing the remainder of the loaner request 240 to be filled. When the manager 250 finishes allocating 330 trays to a loaner request 240, the loaner request 240 is marked as “filled” in order to remove it from the unfilled queue.

In another embodiment of the loaner system, the allocation process is automated by an intelligent suggestion module in which the system automatically retrieves a subset of tray serial numbers that will be allocated to the selected loan types designated in a certain loaner request 240. The allocation manager 250 uses the computing device 9 to invoke the web application 19, thereby obtaining access to the software suite 30 and the loaner management module 38. The allocation manager 250 then uses the loaner management module 38 to electronically select the intelligent suggestion sub-module, such as by clicking an electronic button labeled “suggested trays.” Upon activation, the suggestion module parses the user's loaner inventory listings to locate the best match criteria for the tray types listed in the loaner request 240, and the suggestion module automatically returns suggest trays based on predesignated match criteria. The allocation manager 250 performs the step of medical asset selection 320 by choosing to select the suggested trays, or override the module to make a manual medical asset selection 320. Upon making the final medical asset selection 320, the allocation manager 250 allocates the equipment 330 for use by the representative, and the system communicates this hard allocation to the user's mobile device 15.

In one embodiment, the intelligent suggestion modules pulls trays based on the following hierarchy: (1) tray status, (2) tray usage, and (3) padding. The framework for this module is very flexible and allows for the addition and accommodation of new metrics in future embodiments of the suggestion module.

### VII. Warehouse Module

After a medical asset has been used in a medical procedure, the medical asset must be reconciled to prepare it for the next case. Any lots that were used, depleted, expired, or exhausted during the medical procedure must be replaced. During this process, the tray and all of its constituent lots must be sterilized and resealed in accordance with industry standards, which are often driven by federal regulations such as FDA regulations. To automate tracking of the trays during this process, one embodiment of the system further comprises a warehouse module 110 as an additional module in the web application 19.

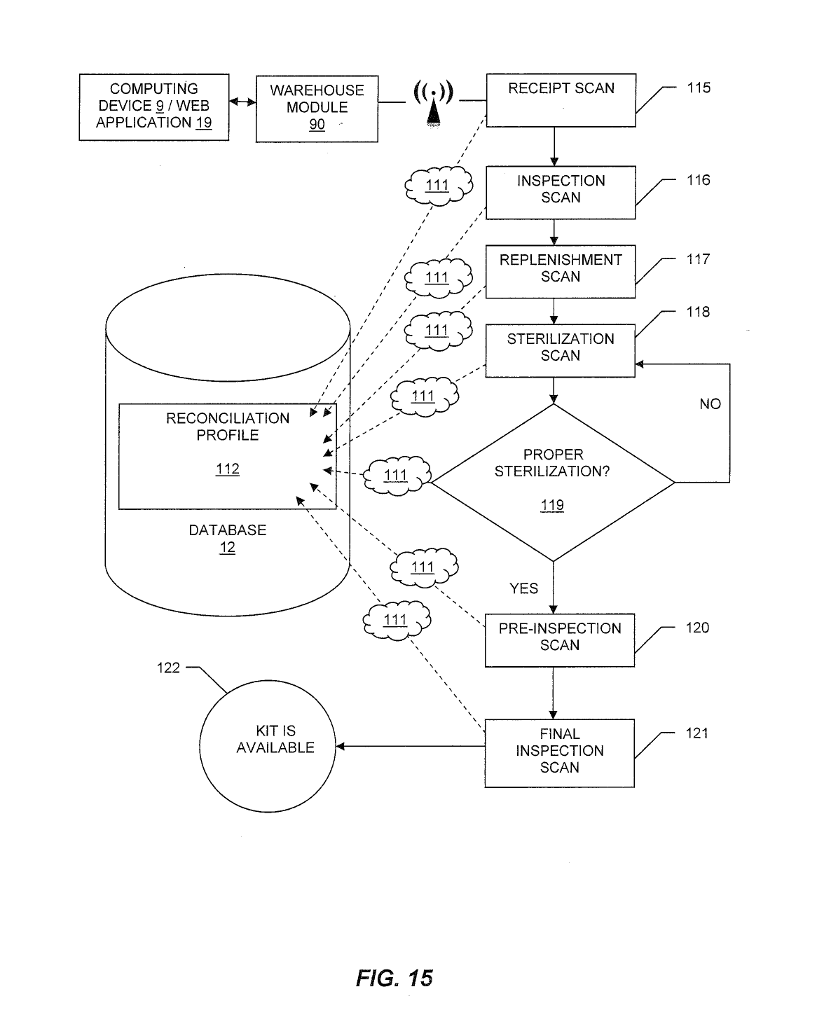

At the conclusion of a case, the representative gathers all trays used in the completed case and sends them to the user's warehouse for the reconciliation process. Referring to FIG. 15, the user then uses the computing device 9 to invoke the web application 19, thereby obtaining access to the software suite 30 and the warehouse module 110 in the software suite 30. The user then uses a scanner to scan an identifier on the tray, thereby performing a receipt scan 115 and logging its arrival at the warehouse. The scanner incorporates one or more of a variety of optical scanning techniques, such as barcodes, QR codes, or the like. The scanner could also be configured to read RFID tags, as an ordinary practitioner will appreciate.

Once the tray is scanned at the warehouse, the status of the tray in the system changes to indicate that the tray has entered the reconciliation process. This indication notifies the user and representatives that the tray is eligible for soft allocation to a later case, but hard allocation is not possible yet.

The tray then moves to an inspection station in the warehouse for the step of an inspection scan 116, which is a scan that indicates the tray's entry into the inspection step. During this step, warehouse personnel inspect the tray, and any lots or other parts that are missing or nonconforming scanned into the system, which communicates in real-time to enterprise resource planning software what is missing. The user uses the warehouse module 110 to electronically enter these deficiencies either manually (typically for missing or untagged lots) or via a scanner (typically for defective or nonconforming lots having a scannable tag or code). The warehouse module 110 communicates this information to the database 122 via the assemblies 13, thereby storing the reconciliation information 91 in the system. The tray then proceeds to the step of inspection where the missing, defective, or nonconforming lots are manually or automatically replaced. During or upon completion of the lot replacement procedure, the tray undergoes an replenishment scan 117, and the data showing replacement of the appropriate lots is added to the reconciliation data 111 and transmitted via the warehouse module 110 to the system, where the data is stored in the database in the reconciliation profile 112.

The tray undergoes a sterilization scan 118 when it begins the sterilization process, which could include sterilization by any known means in the art, such as by autoclave or the like. In the step of sterilization, the tray is disinfected by heating the tray to a threshold sterilizing temperature for a minimum time duration, as is required by relevant industry standards. The next step is a determination of proper sterilization 119. If the tray fails to undergo the required sterilization process, whether because the threshold temperature was not reached or otherwise, the tray is returned to begin the sterilization process again.

Some sterilization equipment has the capability to record wash cycle data corresponding to each wash cycle. For example, the autoclave may have the capability to record wash cycle data such as wash cycle identification number, the maximum temperature reached, whether the threshold sterilization temperature was reached, the time duration that the threshold temperature was reached, the identification of the trays or kits included in the wash cycle, and other information. In these instances, the warehouse module comprises functionality to receive the wash cycle data from the sterilization equipment. For example, in one embodiment the warehouse module is configured to receive wash cycle data from an API built into the sterilization equipment that communicates the wash cycle data gathered by such equipment.

Once the tray is properly sterilized, the tray proceeds to the step of pre-inspection, during which the user performs a pre-inspection scan 120. If the user has not already done so, at this point the user inspects the trays to detect missing, defective, nonconforming, upgraded, improved or other types of new or additional parts. Any such new or additional parts should be sterilized to prevent contamination of the other components of the tray. Upon completion of the lot replacement procedure, as described above, the tray is rescanned and the reconciliation data 111 showing replacement of the appropriate lots is transmitted via the warehouse module 110 to the system, where the data is stored in the database 12 in the reconciliation profile 112.

Once the tray has been sterilized, replenished, and sealed, it is then scanned a final time in the form of a final inspection scan 121, and the warehouse module 110 communicates information to the system indicating that the tray or kit is available for use in a new case 122, as needed. This availability status is logged in the database 12 and indicated in the user's profile 1. The actual location of the tray in the warehouse is logged into the system to provide real-time location visibility to the user. The location may be identified by shelf number, aisle number, or other such location identifying information.

At each scan, the scanned reconciliation data 111 is communicated to the system and stored in the database in a reconciliation profile 112 for each tray. The reconciliation data 111 stored in the reconciliation profile 112 includes one or more of: (i) the time and date that the tray entered and completed each step, (ii) the action that was taken on the tray, such as replacing, removing, or inspecting the lots, (iii) the part numbers, lot numbers, or other identifying numbers of replacement lots introduced into the tray during the reconciliation process, (iv) the batch numbers or other identifiers corresponding to the sterilization batch or other process that the tray underwent, (v) wash cycle data, and (vi) any other relevant information.

For example, after the scanner scans the identifier on the tray and collects the relevant tray reconciliation data 111, the warehouse module 110 communicates the reconciliation data 111 to the database 12 via the assembly 13. This data is then stored in a reconciliation profile 112 for each tray in the user's medical asset inventory listing so that the user and its representatives have access to each tray's historical reconciliation data 111 when needed. Consequently, the system logs complete reconciliation data 111 about the tray at each step of the reconciliation process.

One example of how the reconciliation data 111 is used is when a recall is issued due to an ineffective sterilization batch. If subsequent events indicate that one of the user's sterilization batches was ineffective, the user is able to identify each tray that was included in the ineffective sterilization batch and issue appropriate recalls. The system is configured to issue this recall command in real-time, such as by push notifications to the software application 16 on the representatives mobile device 15. In this manner, a representative can remove a recalled tray from a medical procedure while the case is in progress, thereby enabling up-to-the-minute safety to the patient. The signature data is added to the reconciliation data 111 and stored in the reconciliation profile 112, as described above.

In another embodiment, the warehouse module 110 further comprises an electronic signature component, thereby enabling the user's personnel to sign off at the completion of each step of the process. This electronic signature includes one or more of a graphic verification button on a touch-screen computing device 9, a screen capture signature on a touch screen computing device 9 as described above, checking an electronic box in a mobile computing device 9, or other equivalent method indicating affirmative assent by the user's personnel. The electronic signature component provides a redundant level of verification at each step of the replenishment process.

### VIII. Method of Use

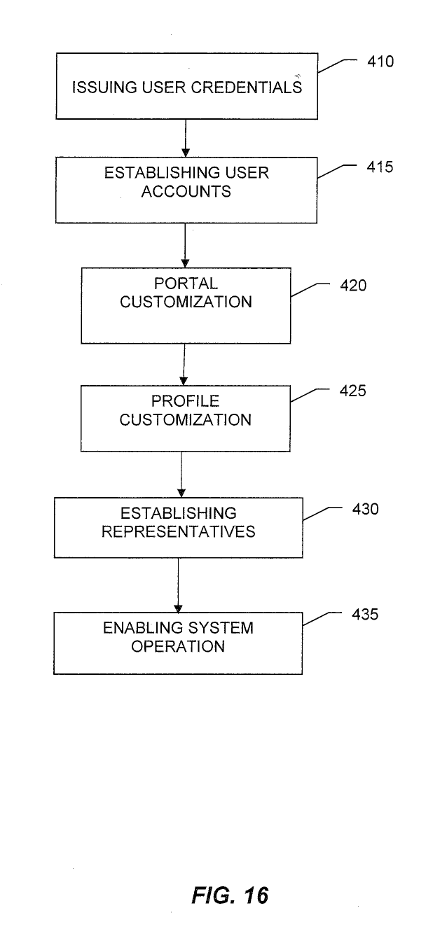

In use, the logistics management system functions as a single, integrated application accessible by the mobile device 15 via the network 17. Referring to FIG. 16, the administrator provides the service to use the system through the steps of issuing user credentials 410, establishing a user account 415, customizing a portal configuration 420, customizing a profile configuration 425, establishing representatives 430, and enabling system operation 435.

In the step of issuing user credentials 410, the administrator generates user credentials for the purposes of user authentication. Each user is assigned a unique set of credentials, which comprises one or more of alphanumeric characters or digital signatures in the form of passwords, user names, or other identifying information. The user must use the credentials to access the system via the website 14. In the step of establishing a user account 415, the administrator collects from the user certain identifying information, which comprises one or more of entity name, address, industry, a description of the user's medical asset inventory, the desired level of service that the user is seeking, and relevant payment information. In the step of customizing a portal configuration 420, the administrator provides the user the opportunity to customize the portal for the website 14 according to the user's medical asset inventory, representatives, unique business structure, desired level of service, and any other factors considered by the user.

In the step of customizing a profile configuration 425, the administrator provides the user the opportunity to customize the user profile 1 according to the user's medical asset inventory, representatives, unique business structure, desired level of service, and any other factors considered by the user. The user profile 1 is organized according to geographic zones, sales locations, healthcare providers (i.e. hospitals or physician practices), representatives, trays, lots, or a variety of other parameters identified by the user. The information submitted by the user is communicated through the website 14 to the database 12 where it is used to populate the user's profile 1. After the initial setup is complete, medical assets or representatives are added or removed from the user's profile 1 by either the user or the administrator. The user designates representatives for removal by modifying a flag on the user's data record, where the flag notifies the administration to remove the representative. In one embodiment, modifying this flag only removes it from view but is not erased from the database 12. The representative's data is erased only with consent of the administrator, thereby reducing instances of fraud, malicious intent, or similar actual or perceived threats.

The data storage within the database 12 table schema follows data normalization, where medical assets, user, and other account data are segregated into separate tables and associated through primary and foreign keys. In one embodiment, user data is communicated via a strict data processing protocol ensuring safety, security, and protection. This secure communication is accomplished by one or more encryption methods, such as by applying, without limitation, PGP, SHA-1, and MD5 encryption standards to the processing of any user-related data. For example, in one embodiment, the data storage occurs on industry proven database platforms on digitally and physically secured servers. The database servers 10, 45 also follow disaster recovery, redundancy, and availability standards that are accepted and approved by certain industries handling sensitive data.

In the step of establishing representatives 430, the administrator designates one or more sales representatives associated with a user, each designated representative having a unique mobile device 15. To activate the representatives, each representative uses the mobile device 15 to retrieve access and account information assigned to the representative by the administrator. In one embodiment, this is accomplished by providing logic in the system that invokes a standard port email transfer of the generated access information to the email provided to the system during the user's setup. In another embodiment, the automated email transfer includes an embedded pass code used by the mobile device 15. The representative then uses the pass code to access the system and download the user application 16 onto the mobile device 15. After initial access of the system, the representative changes the auto-generated pass code to an alphanumeric or digital code unknown to the administrator.

The application 16 then connects to the web service assembly 13 in order to form a communication connection between the mobile device 15 and the tracking system. The application 16 provides the representative access to the features and functionality associated with each of the modules described above. For example, the application 16 presents process indicators for the receiver 21, pin locator 22, inventory viewer 23, case scheduling module 24, case manager 25, optical scanner 26, communicator 27, and preferences reporter 28 modules, among others. Through these indicators, the representative accesses the application 16 and system functions, data, and features available through each respective module.

By way of example, the representative uses the inventory viewer 23 module to retrieve medical asset inventory data for display on the device 15. The medical asset data retrieved and displayed by the device 15 is based on the account setup, in particular pertaining to the geographic location and zone in which the representative is active. The medical asset data, geographic data, and all other data pertinent to the representative are downloaded onto the mobile device 15 as a locally cached file, thus permitting availability of the data in the event of a loss of system connectivity. This file is automatically refreshed to synchronize with the user's medical asset data on the database 12, and the automatic refresh rate can be set to a predetermined time interval, the login of the representative, the restoration of lost connectivity, or a number of other intervals or triggering events. The file could also be automatically refreshed upon changes to medical asset data occurring either on the database 12 or in the locally cached file.

In the step of enabling system operation 435, the administrator provides the representative with the opportunity to establish geographic coordinates of the health care provider, the representative, the trays, or other pertinent information.

In one embodiment of the method of logistics management, the method further comprises providing an RSS feed assembly and functionality. The RSS feed assembly is a live, real-time data and information feed reporting news and releases issued from the FDA or other sources, and the feed is accessible by the user application 16 on the mobile device 15. By accessing and reviewing the feed, the representative can remain appraised of any changes in FDA approval relating to the medical assets managed by the representative. For example, the FDA news feeds can be electronically gleaned for news relating to specific tray or lot numbers, and these numbers can be compared to and synched with the medical asset data in the user's profile 1. Upon locating a medical asset identification number in the database 12 for which an FDA release has been issued, the affected medical asset in the database 12 is flagged to obtain the representative's attention. After seeing a flag associated with a specific tray or lot number, the representative will know that an FDA release has been issued, and the release should be reviewed before using the corresponding medical asset in a medical procedure. When a case is in progress, the FDA flags are communicated real-time via the case manager 25, scheduler 32, or inventory manager 35 so that either the representative, the user, or both, are alerted to the FDA warning. Such real-time alerts during a case allow the representative to prevent a recalled device from being used in a procedure or implanted into a patient as a result of poor information flow.

In one embodiment of the method of logistics management, the method further comprises the step of providing functionality or assemblies that enable the user to pinpoint the representative's geographic location through the handheld devices built-in GPS geo-location. When the healthcare facility receives a shipment of medical assets from a manufacturer, the representative is on location to receive the shipment and pin the location of the trays and lots via the pin locator 22 module. The representative then accompanies the shipment to its physical storage location, such as a storage room or an operating room. The representative is also present in the operating room during the surgery to record the tray and lot numbers of any medical assets depleted during the surgery. Thus, the built in geo-location features in the representative's mobile device 15 serve as an electronic tag to locate the medical assets at any given time after delivery to the healthcare facility. The user sets the location of medical assets by invoking logic that retrieves the current coordinates and other relevant data, and saving the data to the database 12 tables via the communication service 11. This is done by accessing the mobile device's 15 location framework and utilizing its exposed longitude and latitude values and hydrating them into the responsible business object that is embedded in the presentation assembly. To facilitate this functionality, the user application 16 comprises an electronic mapping feature, such as Google® maps, enabling the representative or the user to see the geographic location of the representative, and therefore the medical assets.

In another embodiment of the method of logistics management system, the method additionally comprises the step of enabling the user to manually manipulate the state or status of a tray related to its availability prior to, during, and after surgery.

In the medical asset tracking system, the user's trays or lots are automatically entered into the user's profile 1 upon shipment from the manufacturing facility. While the medical assets are in shipping transit, its location is tracked by gathering and reporting the shipping information available from the courier, most of which report tracking information on various websites. Upon delivery of the medical assets to the healthcare facility, a representative is on location using the mobile device 15 to receive the shipment and log the relevant tracking parameters into the user's profile 1. As the medical assets are stored or maintained at the location of the healthcare facility, the representative uses the mobile device 15 to log any notable changes in the tracking parameters of the medical assets. As described above, the representative is then on location for the surgery where specific lots are depleted via installation into a patient, and the representative uses the mobile device 15 to log this depletion of the specific lot. Thus, after the medical assets are delivered to the healthcare provider, the representative and his or her mobile device 15 is at the location of the medical assets. In this manner, the physical location of the medical assets can be pinned via the GPS location features of the mobile device 15. Additional tracking parameters of the medical assets are also available from the database.

For example, if the FDA issues a health warning or recall of a specific lot, the representative can immediately identify the location of each such lot currently in inventory, in addition to the tracking parameters of each such depleted lot. In embodiments where the representative receives FDA notice real-time, the representative has the ability to notify healthcare providers during a surgical procedure that the lot used in the procedure carries a newly discovered health risk or other defect. In addition, by using the tracking parameters for each depleted lot, the representative can identify the patient in which each lot was installed, the healthcare provider that performed the procedure, and even the manufacturing facility and batch in which the lot was made. Thus, each user, healthcare provider, and patient can minimize health risks or take preventative action by being warned of the danger communicated in the FDA notice or recall. Individual patients are notified that they have received a device that poses certain risks, according to the FDA notice. Otherwise, after the conclusion of a surgery, and hence usage of a tray have concluded, a next surgery is scheduled for the tray, and the representative then uses the mobile device 15 to revise coordinates and enter lot data, tracking parameters, and associated surgery information into the system for real-time retrieval via the mobile device 15 or the website 14.

The embodiments disclosed above are merely representative of the system and method and not meant for the purposes of limitation. One having ordinary skill in the art would understand that the individual features of several disclosed embodiments are interchangeable with the features of other embodiments. For example, the system could comprise additional assemblies or any combination of the assemblies and modules disclosed herein, as desired. Also, multiple formations of the system architecture are a matter of design choice. Consequently, it is understood that equivalents and substitutions for certain elements and components set forth above are part of the invention, and the true scope of the invention is set forth in the claims below.

We claim:

1. A computer-based method of scheduling and tracking medical asset inventory designated for a medical procedure, the method comprising the steps of:
defining a set of case information data comprising:
(i) a list of one or more requested medical assets corresponding to the medical procedure; and
(ii) data corresponding to one or more of a healthcare facility where the medical procedure is to be performed, a patient that is to undergo the medical procedure, a provider that is to perform the medical procedure, a date on which the medical procedure is to be performed, and a time at which the medical procedure is to be performed;
invoking a case scheduling module of a software application supported by a mobile device;
transmitting the case information data to a user profile defined within a remote database by using the case scheduling module to execute a wireless transmission of the case information data from the mobile device to the database, wherein the database is in operative communication with a processor, and wherein the user profile comprises a list of available medical assets;
determining whether the list of available medical assets includes any responsive assets that are responsive to the requested medical assets for the medical procedure, wherein the determination is made automatically by the processor;
identifying one or more on-site responsive medical assets that are currently located at the location where the medical procedure is to be performed, wherein the identification is made automatically by the processor; and
allocating one or more of the identified on-site responsive assets to the medical procedure, wherein the allocation is made automatically by the processor.

2. The method of claim 1, wherein the step of identifying one or more on-site responsive medical assets further comprises the step of identifying one or more off-site responsive medical assets that are not currently located at the location where the medical procedure is to be performed, wherein the identification of the one or more off-site responsive medical assets is made automatically by the processor.

3. The method of claim 2, further comprising the step of calculating, based on the current location of the identified off-site responsive medical assets, the shipping time required for delivery of each of the identified off-site responsive medical assets to the location where the medical procedure is to be performed, wherein the calculation is made automatically by the processor.

4. The method of claim 3, further comprising the step of determining, based on the calculated shipping time, whether any of the identified off-site responsive medical assets can fill the list of requested medical assets, wherein the determination is made automatically by the processor.

5. The method of claim 4, further comprising the step of allocating one or more of the identified off-site responsive assets to the medical procedure when such identified off-site responsive assets is determined to be deliverable in a timely manner, wherein the allocation is made automatically by the processor.

6. The method of claim 4, further comprising the step of invoking a loaner request module of the software application supported by the mobile device when none of the identified off-site responsive medical assets is determined to be deliverable in a timely manner to the location where the case is to be performed.

7. An apparatus comprising:
at least one processor; and
at least one memory device having computer program instructions, the at least one memory and the computer program instructions being configured in cooperation with the at least one processor to cause the apparatus to:
define a set of case information data comprising:
(i) a list of requested medical assets corresponding to the medical procedure; and
(ii) data corresponding to one or more of a healthcare facility where the medical procedure is to be performed, a patient that is to undergo the medical procedure, a provider that is to perform the medical procedure, a date on which the medical procedure is to be performed, and a time at which the medical procedure is to be performed;
invoke a case scheduling module of a software application supported by a mobile device;
transmit the case information data to a user profile defined within a remote database by using the case scheduling module to execute a wireless transmission of the case information data from the mobile device to the database, wherein the database is in operative communication with a processor, and wherein the user profile comprises a list of available medical assets;
determine whether the list of available medical assets includes any responsive assets that are responsive to the requested medical assets for the medical procedure, wherein the determination is made automatically by the processor;
identify on-site responsive medical assets that are currently located at the location where the medical procedure is to be performed, wherein the identification is made automatically by the processor; and
allocate the identified on-site responsive assets to the medical procedure, wherein the allocation is made automatically by the processor.

8. The apparatus of claim 7, wherein the at least one memory device and the computer program instructions are configured to, in cooperation with the at least one processor, cause the apparatus to identify one or more on-site responsive medical assets further comprises the step of identifying one or more off-site responsive medical assets that are not currently located at the location where the medical procedure is to be performed, wherein the identification of the one or more off-site responsive medical assets is made automatically by the processor.

9. The apparatus of claim 8, wherein the at least one memory device and the computer program instructions are configured to, in cooperation with the at least one processor, cause the apparatus to calculate, based on the current location of the identified off-site responsive medical assets, the shipping time required for delivery of each of the identified off-site responsive medical assets to the location where the medical procedure is to be performed, wherein the calculation is made automatically by the processor.

10. The apparatus of claim 9, wherein the at least one memory device and the computer program instructions are configured to, in cooperation with the at least one processor, cause the apparatus to determine, based on the calculated shipping time, whether any of the identified off-site responsive medical assets can fill the list of requested medical assets, wherein the determination is made automatically by the processor.

11. The apparatus of claim 10, wherein the at least one memory device and the computer program instructions are configured to, in cooperation with the at least one processor, cause the apparatus to allocate one or more of the identified off-site responsive assets to the medical procedure when such identified off-site responsive assets is determined to be deliverable in a timely manner, wherein the allocation is made automatically by the processor.

12. The apparatus of claim 10, wherein the at least one memory device and the computer program instructions are configured to, in cooperation with the at least one processor, cause the apparatus to invoke a loaner request module of the software application supported by the mobile device when none of the identified off-site responsive medical assets is determined to be deliverable in a timely manner to the location where the case is to be performed.
